(19) United States
(12) Patent Application Publication
(10) Pub. No.: US 2024/0373887 A1
(43) Pub. Date: Nov. 14, 2024

(54) CELL POWDER TYPE CULTURED MEAT AND ITS MANUFACTURING METHOD

(71) Applicant: UIF (UNIVERSITY INDUSTRY FOUNDATION), YONSEI UNIVERSITY, Seoul (KR)

(72) Inventors: Jinkee HONG, Seoul (KR); Bumgyu CHOI, Seoul (KR)

(73) Assignee: UIF (UNIVERSITY INDUSTRY FOUNDATION), YONSEI UNIVERSITY, Seoul (KR)

(21) Appl. No.: 18/659,583

(22) Filed: May 9, 2024

(30) Foreign Application Priority Data
May 9, 2023 (KR) 10-2023-0059602
Jul. 14, 2023 (KR) 10-2023-0091442

(51) Int. Cl. A23L 13/10; C12N 5/00; C12N 5/071; C12N 5/077; C12N 5/0775

(52) U.S. Cl. CPC A23L 13/10; C12N 5/0062; C12N 5/0658; C12N 5/0667; C12N 5/0692; C12N 2506/1338; C12N 2506/1384; C12N 2506/28; C12N 2513/00

(57) ABSTRACT

The present disclosure is directed to providing a method for preparing powdered cultured meat, which can increase the efficiency of cell differentiation and the protein content of cultured meat with economic feasibility and to providing powdered cultured meat with high physicochemical stability and transportability and a food composition containing the same. The powdered cultured meat has a flavor similar to that of real meat, and can be highly utilized as a high-protein food in a variety of food compositions or processed foods.

### CROSS-REFERENCE TO RELATED APPLICATION

This application claims priority to Korean Patent Application Nos. 10-2023-0059602 and 10-2023-0091442, filed on May 9, 2023 and Jul. 14, 2023 respectively and all the benefits accruing therefrom under 35 U.S.C. § 119, the contents of which in its entirety are herein incorporated by reference.

### BACKGROUND

### 1. Field

The present disclosure relates to a method for preparing powdered cultured meat, powdered cultured meat prepared using the same, and a food composition containing the same.

### 2. Description of the Related Art

With the problems of global population growth, environmental challenges and limited resources, meat substitute technologies are being developed to address environmental and animal welfare issues. Various protein sources are being developed, ranging from plant-based meat substitutes to proteins extracted from microorganisms, microalgae, insects, etc., and interest in meat substitutes as future food is increasing. In particular, research on cultured meat, which is cell-based meat as a sustainable food resource, is being conducted actively.

Cultured meat refers to edible meat obtained through cell proliferation by cell engineering technology by cultivating cells of living animals in a laboratory without going through the process of raising livestock. It is also called in-vitro meat or lab-grown meat in that it is grown in a test tube, artificial meat in that it is synthesized by humans using stem cells, clean meat in that it is produced in a clean production facility rather than the traditional breeding facility, and bio-artificial muscles (BAMs) in that muscle fibers that make up the cultured meat are cultivated.

The most important factor in the commercialization of cultured meat is the expansion of the scale of production. Cultured meat is generally produced by extracting cells from livestock, proliferating them in large quantities, differentiating them on a scaffold, and processing them into a final product. In particular, the scaffold is an essential element for mass production of cultured meat and food. However, unlike the scaffolds used in tissue engineering, the development of scaffolds for cultured meat requires the use of only food-grade materials and the approval from agencies such as the FDA. In addition, the cost of developing the scaffolds remains a major obstacle to the commercialization of cultured meat.

As a result, there is a need for a cultured meat manufacturing method which has high price competitiveness and can be highly utilized as food.

### SUMMARY

The present disclosure is directed to providing a method for preparing powdered cultured meat, which can increase the efficiency of cell differentiation and the protein content of cultured meat with economic feasibility.

The present disclosure is also directed to providing powdered cultured meat with high physicochemical stability and transportability, and a food composition containing the same.

A method for preparing powdered cultured meat according to the present disclosure may include: a cell proliferation step of proliferating cells in a proliferation culture medium containing 0.1 wt % or more and less than 10 wt % of serum based on the total weight of the proliferation culture medium; a differentiation step of differentiating the proliferated cells; a cell powdering step of powdering the differentiated cells.

The cells may be selected from adipose stem cells, muscle stem cells, and vascular stem cells.

The cell powdering step may include collecting the differentiated cells and lyophilizing them.

The serum may be fetal bovine serum.

In the cell differentiation step, the fusion index of the cells may be 40% or higher.

The present disclosure may provide powdered cultured meat prepared through the method for preparing powdered cultured meat according to an exemplary embodiment of the present disclosure.

The powdered cultured meat may contain 30 wt % or more of proteins based on total weight.

The powdered cultured meat according to the present disclosure may include first powdered cultured meat cultured from any stem cells selected from a group consisting of adipose stem cells, muscle stem cells and vascular stem cells, and second powdered cultured meat cultured from stem cells different from the stem cells of the first cultured meat, wherein the first powdered cultured meat and the second powdered cultured meat are mixed with each other in a powdered form, and the first powdered cultured meat and the second powdered cultured meat are prepared through the method for preparing powdered cultured meat described above.

The powdered cultured meat may include 10 to 45 wt % of adipose stem cell-derived powdered cultured meat and 55 to 90 wt % of muscle stem cell-derived powdered cultured meat.

The present disclosure may provide a food composition containing the powdered cultured meat according to an exemplary embodiment of the present disclosure.

The present disclosure may provide a specialized processed food containing the powdered cultured meat according to an exemplary embodiment of the present disclosure.

The specialized processed food may be space food, C-ration, medical food, or leisure food.

The method for preparing powdered cultured meat according to an exemplary embodiment of the present disclosure is cost-effective, and can increase the protein content of cultured meat by promoting cell differentiation.

Powdered cultured meat according to an exemplary embodiment of the present disclosure has a flavor similar to that of real meat, and can be highly utilized as a high-protein food in a variety of food compositions or processed foods.

### BRIEF DESCRIPTION OF THE DRAWINGS

FIG. 1 schematically shows a method for preparing powdered cultured meat according to the present disclosure.

FIG. 2 schematically shows cell proliferation and differentiation processes in a method for preparing powdered cultured meat of the present disclosure.

FIG. 3 shows cell proliferation rate measured using Cell Counting Kit-8 during a cell proliferation.

FIG. 4 shows the morphology of differentiated cells imaged using a confocal microscope (CLSM; LSM 880, Carl Zeiss).

FIG. 5 shows the protein content and fusion index of differentiated cells measured using a bicinchoninic acid assay (BCA) kit.

FIG. 6 shows the flavor composition of powdered cultured meat measured by gas chromatography-mass spectrometry (GC-MS).

FIG. 7 shows the flavor comparison of the powdered cultured meat and grilled meat.

### DETAILED DESCRIPTION

The present disclosure is described in detail below. Unless defined otherwise, the terms used in this specification should be construed as generally understood by those who have ordinary knowledge in the art. The drawings and exemplary embodiments of this specification are intended to make it easy for those having ordinary skill to understand and implement the present disclosure. The contents that may obscure the gist of the present disclosure may be omitted from the drawings and exemplary embodiments, and the present disclosure is not limited to the drawings and exemplary embodiments.

As used herein, singular forms are intended to include p forms as well, unless the context clearly dictates otherwise.

Numerical ranges used herein include the lower and upper limits and all values within that ranges, increments logically derived from the ranges being defined, all doubly defined values, and all possible combinations of the upper and lower limits of the numerical ranges defined in different forms. Unless defined otherwise in this specification, values outside the numerical ranges that may occur due to experimental error or rounding of values are also included in the defined numerical ranges.

The terms include, have, possess, etc. used in this specification indicate the existence of the features or components described in the specification and do not preclude the possibility of the addition of one or more other features or components, unless specified otherwise.

The present disclosure provides a method for preparing powdered cultured meat, which can increase the protein content of cultured meat due to high cell differentiation rate and is cost-effective.

The method for preparing powdered cultured meat according to an exemplary embodiment of the present disclosure may include: a cell proliferation step of proliferating cells in a proliferation culture medium containing 0.1 wt % or more and less than 10 wt % of serum based on the total weight of the proliferation culture medium; a differentiation step of differentiating the proliferated cells; a cell powdering step of powdering the differentiated cells.

Specifically, the proliferation culture medium may contain 0.1 wt % or more and less than 9 wt %, more specifically 1 wt % or more and less than 8 wt %, of serum based on the total weight of the proliferation culture medium.

In the existing methods for preparing cultured meat, the composition of a differentiation culture medium is changed to increase the differentiation rate of cells. However, since the enzymes and signaling materials that promote differentiation added to the culture medium are expensive, the price competitiveness of cultured meat is decreased significantly. Accordingly, the present disclosure solves the above problem and provides a method for preparing powdered cultured meat that can promote differentiation only by changing the composition of a proliferation culture medium. Specifically, the method for preparing powdered cultured meat of the present disclosure can provide the effect of improving the differentiation efficiency of cells and increasing the protein content of cultured meat economically without further change in the composition of the culture medium by replacing the high-concentration serum contained in the existing proliferation culture medium with a low-concentration serum.

Serum is known to promote cell proliferation. The proliferation culture medium used in the method for preparing cultured meat generally contains high-concentration serum of at least 10%. However, the inventors of the present disclosure completed the present disclosure by finding that the cells proliferated under the condition of low serum concentration of lower than 10% show a high differentiation rate in the differentiation step after the proliferation step.

In addition, since the method for preparing powdered cultured meat of the present disclosure prepares meat without using a scaffold, it is highly price-competitive, and the physicochemical stability of the cultured meat can be improved through the cell powdering step.

According to an exemplary embodiment, the serum may be fetal bovine serum. Since fetal bovine serum (FBS) has a smaller amount of antibodies and contains more growth factors than ordinary serum, it is effective for cell culture and is less likely to trigger immune responses in various cells.

According to an exemplary embodiment, the cells may be selected from a group consisting of chondrocytes, fibrochondrocytes, osteocytes, osteoblasts, osteoclasts, synoviocytes, myelocytes, neurons, adipocytes, mesenchymal cells, epithelial cells, hepatocytes, muscle cells, stromal cells, vascular cells, stem cells, embryonic stem cells, mesenchymal stem cells, progenitor cells derived from adipose tissue, peripheral blood progenitor cells, stem cells isolated from adult tissue, and induced pluripotent stem cells (iPS cells), specifically stem cells derived from adipose tissue, muscle tissue or vascular tissue, more specifically muscle stem cells, although the cells are not limited as long as they can be used to prepare cultured meat.

In addition, the cells may be taken from cattle, pigs, chickens, goats, sheep or ducks, specifically from cattle, pigs or chickens, although not being limited thereto.

The cell powdering step may include collecting the differentiated cells and lyophilizing them. The lyophilization may be carried out below −10° C. for 10 hours or longer, specifically at −10 to −50° C. for 10 to 50 hours.

In the cell differentiation step, the fusion index of the cells may be 40% or higher, specifically 50% or higher. The fusion index is an index that can predict the degree of differentiation of cells, and it can be calculated by dividing the number of nuclei in myotubes having two or more myonuclei observed with a microscope by the total number of the nuclei, according to the following Equation 1.

$$Fusion index = \frac{The number of nuclei in myotube ( \geq 2 myonuclei )}{The total number of nuclei} \times 100 \qquad [ Equation 1 ]$$

The present disclosure provides powdered cultured meat prepared through the method for preparing powdered cultured meat according to an exemplary embodiment of the present disclosure. The powdered cultured meat can be usefully utilized as food because it has a superior protein content and the ingredients of the cultured meat can be easily mixed one another.

The powdered cultured meat may contain 30 wt % or more, specifically 40 wt % or more, of proteins based on total weight. The protein content of the powdered cultured meat is higher than that of chicken breast and beef tenderloin, which are known as high-protein foods, showing that the powdered cultured meat can be used as an efficient high-protein food.

The powdered cultured meat of the present disclosure may include first powdered cultured meat cultured from any stem cells selected from a group consisting of adipose stem cells, muscle stem cells and vascular stem cells, and second powdered cultured meat cultured from stem cells different from the stem cells of the first cultured meat, wherein the first powdered cultured meat and the second powdered cultured meat are mixed with each other in a powdered form, and the first powdered cultured meat and the second powdered cultured meat are prepared through the method for preparing powdered cultured meat described above.

The powdered cultured meat may be prepared by mixing the first powdered cultured meat to n-th cultured meat prepared through the method for preparing powdered cultured meat according to an exemplary embodiment of the present disclosure. Since the nutrient content of the cultured meat can be controlled by simply mixing powdered cultured meat produced from different stem cells at a desired ratio, it is valuable in terms of the convenience in the preparation process and as food. The n may be 2 to 10, specifically 2 to 5, although not being limited thereto.

The powdered cultured meat may include 10 to 45 wt % of adipose stem cell-derived powdered cultured meat and 55 to 90 wt % of muscle stem cell-derived powdered cultured meat, specifically 15 to 45 wt % of adipose stem cell-derived powdered cultured meat and 55 to 75 wt % of muscle stem cell-derived powdered cultured meat, more specifically 30 to 40 wt % of adipose stem cell-derived powdered cultured meat and 60 to 70 wt % of muscle stem cell-derived powdered cultured meat. Since 100 g of beef tenderloin contains about 26.5 g of proteins and about 17.5 g of fats, the powdered cultured meat may have a flavor similar to that of real meat when it satisfies the above-described w % ranges, without being limited to specific parts of beef.

The present disclosure provides a food composition containing the powdered cultured meat according to an exemplary embodiment of the present disclosure. In an exemplary embodiment, the food composition may be one or more selected from a group consisting of snacks, dumplings, fried foods, stir-fries, sauces, seasonings, powder mixes, breads, beverages, processed canned foods, dried seaweeds and processed noodles, and the form added to the food may be ground into various particle sizes depending on the purpose for which it is used in the food. The grinding for addition to food may be uniform or nonuniform with an average size of 1 μm to 10 cm, specifically 0.1 μm to 10 mm or 0.5 μm to 5 mm, more specifically 1 μm to 1 mm.

The present disclosure provides a specialized processed food including the powdered cultured meat according to an exemplary embodiment of the present disclosure. The specialized processed food may be space food, C-ration, medical food, or leisure food, specifically a convenience food such as meal replacement, emergency food in case of disaster or emergency, and portable food for outdoor activities such as mountain climbing or fishing, although not being limited thereto.

The specialized processed food may be a high-protein, high-nutrient product including the powdered cultured meat of the present disclosure. Since it is in powder form and can be easily vacuum-packed in a sterile state, it can be stored for a long time and can be eaten easily even in an extreme environment without water, fire and cooking utensils.

Hereinafter, the method for preparing powdered cultured meat according to the present disclosure and the powdered cultured meat prepared using the same will be explained in more detail through specific examples. However, the following examples are only examples for describing the present disclosure in detail, and the present disclosure can be implemented in various forms without being limited thereto. In addition, the terms used in the description of the present disclosure are intended only to effectively describe the specific examples and are not intended to limit the present disclosure.

### [Example 1] Preparation of Powdered Cultured Meat

### Cell Proliferation and Differentiation Steps

Mouse embryonic myoblast C2C12 cells as precursors of skeletal muscle cells obtained from the American Type Culture Collection (ATCC) were seeded in a culture dish at a density of 2×10³ cells/cm² and then cultured for 7 days in a DMEM medium (proliferation culture medium) containing 5% fetal bovine serum (FBS) and 1% penicillin/streptomycin (PS) antibiotics. The culturing was conducted in a humidified atmosphere of 37° C. and 5% CO₂. After 7 days of culturing, when the cells in the medium reached 100% confluency, the medium was replaced with a differentiation culture medium containing 5% horse serum (HS; Thermo Fisher Scientific) and 1% PS, and the cells were differentiated for 5 days. All the media used for culturing were replaced every two days.

### Cell Powdering Step

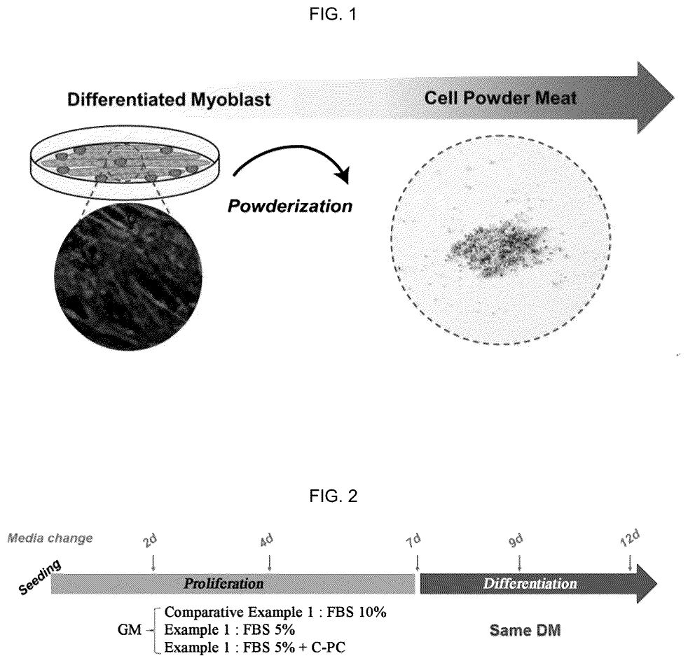

After the C2C12 myoblasts were cultured for 12 days through the cell proliferation and differentiation steps, the medium was removed and the cells were washed with 1x phosphate-buffered saline (PBS). Afterwards, the cells were carefully separated from the culture dish using a cell scraper, and the separated and unseparated cell masses were collected in distilled water and transferred to a 1.5-mL microtube. The microtube was centrifuged at 300×g for 2 minutes to form pellets, and after removing the supernatant, the pellets were pre-frozen at −20° C. for 12 hours and then lyophilized for additional 24 hours. The cell culture condition and the powdered cultured meat preparation process are shown in FIGS. 1 and 2.

### Example 2

Experiment was conducted in the same way as Example 1, except that 50 μg/mL C-phycocyanin (C-PC, Sigma-Aldrich) was added to the proliferation culture medium of Example 1.

### Example 3

Experiment was conducted in the same way as Example 1, except that a DMEM medium containing 2% fetal bovine serum (FBS) was used as the proliferation culture medium of Example 1.

### Example 4

Experiment was conducted in the same way as Example 1, except that a DMEM medium containing 7% fetal bovine serum (FBS) was used as the proliferation culture medium of Example 1.

### Comparative Example 1

Experiment was conducted in the same way as Example 1, except that a DMEM medium containing 10% fetal bovine serum (FBS) was used as the proliferation culture medium of Example 1.

### [Evaluation Example 1] Characterization of Cultured Cells (1)-Cell Proliferation Rate

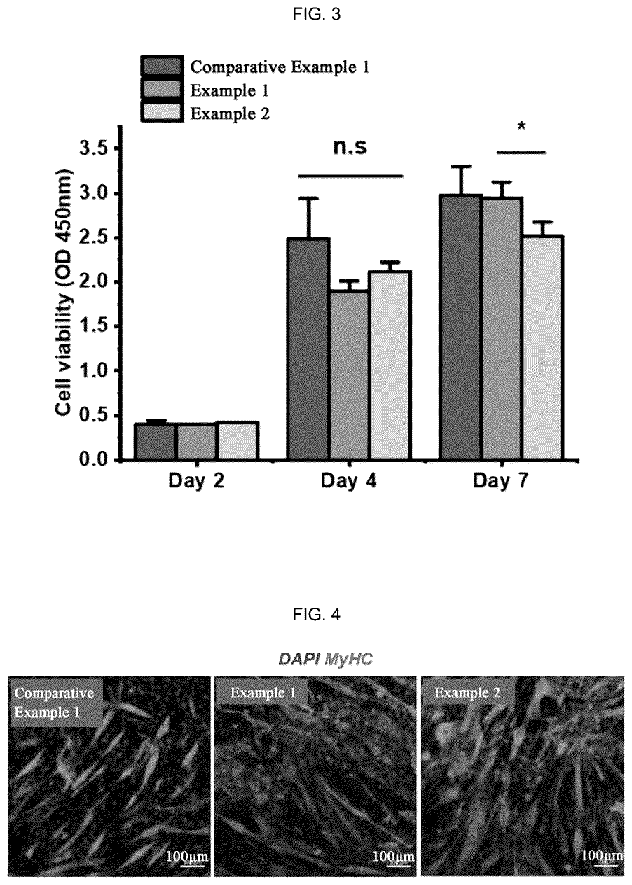

In order to evaluate cell proliferation rate, the amount of the cells cultured for 7 days in the proliferation culture media of Examples 1 and 2 and Comparative Example 1, respectively, was measured using Cell Counting Kit-8 (CCK-8, D-Plus CCK cell viability assay kit, Dongin LS, Korea). The result is shown in FIG. 3.

As shown in FIG. 3, Comparative Example 1 with a high FBS content showed high cell proliferation rate until day 4, but there was no significant difference. It can be seen that the cell proliferation rate on day 7 was similar in all the three groups of Examples 1 and 2 and Comparative Example. Although the number of cells was slightly higher in Comparative Example 1 on average during the proliferation process as compared to Examples 1 and 2, there was no significant difference.

### [Evaluation Example 2] Characterization of Cultured Cells (2)-Cell Differentiation Rate

### Immunofluorescence Staining

To evaluate cell differentiation rate, the C2C12 cells for a total of 12 days in the proliferation culture media and differentiation culture media of Examples 1 and 2 and Comparative Example 1, respectively, were washed twice with 1×PBS, fixed in formaldehyde solution (Sigma-Aldrich) at room temperature for 15 minutes, and washed twice more with PBS. To prevent non-specific protein binding, the cells were cultured at 4° C. in a blocking medium containing 2% (v/v) bovine serum albumin (BSA), 0.3% (v/v) Triton X-100, 10% (v/v) horse serum and PBS. The cells were then cultured for 2 hours at room temperature in a medium containing myosin heavy chain (MyHC) antibody MF20 and 1×PBS diluted 100-fold with a 2% (v/v) BSA, 10% (v/v) HS solution. After the culturing, the cells were washed once with PBS and once with 0.025% (v/v) Triton X-100, and then treated with Alexa Flour 594-conjugated donkey anti-mouse IgG as a secondary antibody, diluted 400-fold with the same diluent as described above, at room temperature for 30 minutes. After washing once with PBS and then with 0.025% Triton X-100, the cells were stained for 30 minutes with the fluorescent dye 4′,6-diamidino-2-phenylindole (DAPI), which was diluted 250-fold with 1% (v/v) BSA solution.

### Measurement of Cell Differentiation Rate

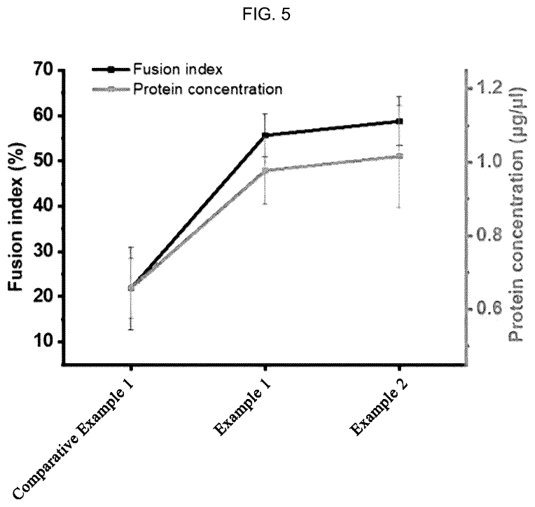

For analysis of myotube formation by the myoblasts, the immunofluorescence-stained cells were subjected to confocal microscopy (CLSM; LSM 880, Carl Zeiss) using a ×10 objective lens. The result is shown in FIG. 4. The cell nuclei were stained blue by DAPI, and the myotube cells were stained red due to the presence of MyHC and Alexa Flour 594. The total number of cells in the 850.19 μm×850.19 μm images was indicated by the number of DAPI-stained nuclei. And, the number of cells in the myotube was indicated by the number of nuclei in the MyHC-stained area. At least three random fields were captured for each sample, and the average value was used to calculate the fusion index as the number of nuclei in the myotube with two or more nuclei divided by the total number of nuclei. The result is shown in Table 1 and FIG. 5.

$$Fusion index = \frac{The number of nuclei in myotube ( \geq 2 myonuclei )}{The total number of nuclei} \times 100 \qquad [ Equation 1 ]$$

TABLE 1

| | Example 1 | Example 2 | Comparative Example 1 |
|---|---|---|---|
| Fusion index | 55.6% | 58.8% | 21.9% |

As shown in Table 1, unlike the result for cell proliferation rate, Examples 1 and 2 wherein the proliferation culture media with low FBS contents were used showed high fusion indices, while Comparative Example 1 wherein the proliferation culture medium with a high FBS content was used showed a low fusion index. Accordingly, it can be seen that the differentiation efficiency of the myoblasts cultured under low serum conditions during the cell proliferation step is increased significantly in the cell differentiation step.

### [Evaluation Example 3] Characterization of Cultured Cells (3)-Protein Content

The C2C12 cells separated in Examples 1 and 2 and Comparative Example were lysed in a radioimmunoprecipitation assay (RIPA) lysis buffer at 4° C. for 30 minutes before powdering and then centrifuged at 10,000×g for 10 minutes to extract proteins from the supernatant. The extracted proteins were quantified with a bicinchoninic acid assay (BCA) kit (Thermo Fisher Scientific), and the result is shown in FIG. 5.

From FIG. 5, it can be seen that the sample exhibiting a higher differentiation rate exhibits a higher protein content. It is thought that the protein content increases as the differentiation rate is higher because proteins such as MyoD, myogenin, troponin T and MHC are expressed during the differentiation of myoblasts.

### [Evaluation Example 4] Characterization of Powdered Cultured Meat (1)-Protein Content

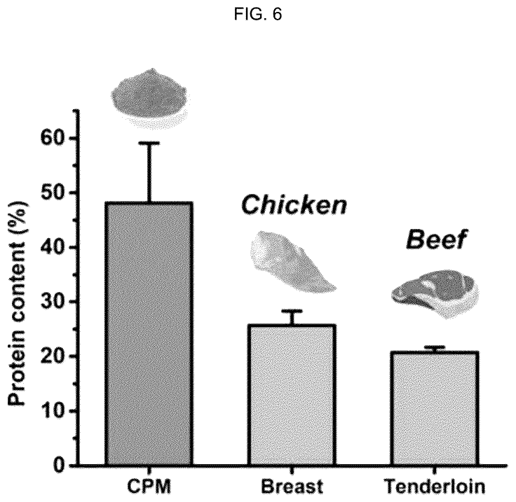

The powdered cultured meat of Example 1, chicken breast and beef tenderloin were lysed in a radioimmunoprecipitation assay (RIPA) lysis buffer at 4° C. for 30 minutes and centrifuged at 10,000×g for 10 minutes. Then, proteins were extracted from the supernatant. The extracted proteins were quantified with a bicinchoninic acid assay (BCA) kit (Thermo Fisher Scientific). The protein content of each sample is shown in FIG. 6.

For Example 1, 1.6 mg of powdered cultured meat was obtained per a 90-mm dish average, and it contained 749.5 μg of proteins on average. Accordingly, as shown in FIG. 6, the powdered cultured meat of Example 1 had a high protein content of 48.1%, which was much higher than that of chicken breast (25.7%) and beef tenderloin (20.7%) generally known as high-protein foods, suggesting that it is a much better source of proteins.

### [Evaluation Example 5] Characterization of Powdered Cultured Meat (2)-Economic Feasibility

### Preparation Cost

The cost efficiency of the powdered cultured meat was evaluated by calculating the cost of the media consumed during cell culture in Examples 1 and 2 and Comparative Example 1. For preparation of the powdered cultured meat per a 90-mm dish, 24 mL of the proliferation culture medium and 16 mL of the differentiation culture medium were required. Accordingly, the cost of the culture media (40 mL in total) was calculated for Examples 1 and 2 and Comparative Example 1. The result is shown in Table 2.

TABLE 2

| | Comparative Example 1 | | Example 1 | | Example 2 | |
|---|---|---|---|---|---|---|
| DMEM | $2.7/45 | mL | $2.9/47.5 | mL | $2.9/47.5 | mL |
| FBS | $4.5/5 | mL | $2.2/2.5 | mL | $2.2/2.5 | mL |
| C-PC | 0 | | 0 | | $50.9/2.5 | mg |
| PS | $0.2/0.5 | mL | $0.2/0.5 | mL | $0.2/0.5 | mL |
| Growth | $7.5/50 | mL | $5.4/50 | mL | $56.3/50 | mL |
| medium | | | | | | |
| HS | $0.4/2.5 | mL | $0.4/2.5 | mL | $0.4/2.5 | mL |
| DM (horse | $3.6/50 | mL | $3.6/50 | mL | $3.6/50 | mL |
| serum 5%) | | | | | | |
| Total | $4.7/40 | mL | $3.7/40 | mL | $28.2/40 | mL |

### Cost Efficiency

The cost efficiency of the powdered cultured meat was calculated by dividing the protein concentration of Examples 1 and 2 and Comparative Example 1 measured in Evaluation Example 4 by the price of each culture medium. The result is shown in Table 3 below. The protein concentration measured in Evaluation Example 4 was the highest for Example 2 (1.3 μg/μL), followed by Example 1 (1.0 μg/μL) and Comparative Example 1 (0.73 μg/μL). However, since the price of the media increased in the order of Example 1, Comparative Example 1 and Example 2, the cost efficiency was 1.76 for Example 1, 1.00 for Comparative Example 1, and 0.31 for Example 2. Considering that the most important factor in cultured meat is price competitiveness, it can be seen that the media of Example 1 are the most suitable for powdered cultured meat because they allow the production of a large amount of proteins at the lowest cost.

TABLE 3

| | Comparative | | |
|---|---|---|---|
| | Example 1 | Example 1 | Example 2 |
| Price ($) | 4.7 | 3.7 | 28.2 |
| Price ratio | 1.0 | 0.8 | 6.0 |
| Protein concentration | 0.729 | 1.008 | 1.329 |
| (μg/μL) | | | |
| Protein ratio | 1.0 | 1.4 | 1.8 |
| Cost efficiency | 1.0 | 1.76 | 0.31 |

### [Evaluation Example 6] Characterization of Powdered Cultured Meat (3)-Flavor

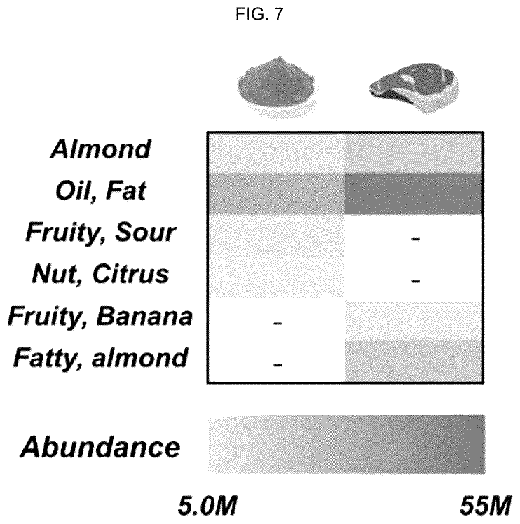

In order to evaluate the potential of the powdered cultured meat as a food, the powdered cultured meat of Example 1 and freeze-dried beef tenderloin were respectively roasted on a hot plate with oil at 120° C. for 15 minutes. Flavor analysis was performed by the headspace solid-phase microextraction (HS-SPME) method of gas chromatography-mass spectrometry (GC-MS, Agilent 8890 GC system-Agilent 5677B MSD, Agilent Technologies). The analytes were separated in an HP-5 ms column (30 m×250 μm×0.25 μm). Each sample was placed in an oven for flavor analysis. The temperature of the oven was initially maintained at 40° C. for 5 minutes, then raised to 160° C. at a rate of 4° C./min, then increased to 250° C. at a rate of 7° C./min, and maintained for 10 minutes. The mass spectra (MS) were acquired in normal scanning mode with a source temperature of 230° C. The volatile compounds generated due to the high temperature were identified by comparison with the data from a spectral library (Agilent Chemstation Integrator), and the flavors were referenced from the Flavor Extract Manufacturers Association of the United States (FEMA) and the Joint FAO/WHO Expert Committee on Food Additives (JECFA). The result is shown in FIG. 7. The color intensity shown in FIG. 7 is proportional to the peak area of the volatile compounds analyzed by GC-MS.

From FIG. 7, it can be seen that both Example 1 and beef contain pentanal, which provides an almond flavor, and hexanal, which provides an oily fatty flavor. Meanwhile, acetic acid, which provides fruity and sour flavors, along with heptanal, which provides fatty and nutty flavors, were detected for Example 1, whereas 2-methylbutyl acetate, which provides fruity flavor, and 3-methylbutanal, which provides fatty and almond flavors, were detected for beef. These results show that the powdered cultured meat of the present disclosure has a nutty flavor similar to that of grilled meat.

Although the present disclosure has been described by specific exemplary embodiments and the limited examples and comparative examples, they are provided only for a more general understanding of the present disclosure, and the present disclosure is not limited to the examples described above. Those having ordinary knowledge in the art to which the present disclosure belongs can make various modifications and modifications based on the description.

Accordingly, the scope of the present disclosure should not be limited to the described exemplary embodiments, and the scope of the appended claims described below as well as all modifications that are equivalent to the scope of the claims shall fall within the scope of the present disclosure.

What is claimed is:

1. A method for preparing powdered cultured meat, comprising:
a cell proliferation step of proliferating cells in a proliferation culture medium containing 0.1 wt % or more and less than 10 wt % of serum based on the total weight of the proliferation culture medium;
a cell differentiation step of culturing the proliferated cells in a differentiation culture medium; and
a cell powdering step of powdering the differentiated cells.

2. The method for preparing powdered cultured meat according to claim 1, wherein the cells are selected from adipose stem cells, muscle stem cells and vascular stem cells.

3. The method for preparing powdered cultured meat according to claim 1, wherein the cell powdering step comprises collecting the differentiated cells and lyophilizing them.

4. The method for preparing powdered cultured meat according to claim 1, wherein the serum is fetal bovine serum.

5. The method for preparing powdered cultured meat according to claim 1, wherein, in the cell differentiation step, the fusion index of the cells is 40% or higher.

6. Powdered cultured meat prepared through the method for preparing powdered cultured meat according to claim 1.

7. The powdered cultured meat according to claim 6, which comprises 30 wt % or more of proteins based on the total weight of the powdered cultured meat.

8. Powdered cultured meat, comprising first powdered cultured meat cultured from any stem cells selected from a group consisting of adipose stem cells, muscle stem cells and vascular stem cells, and second powdered cultured meat cultured from stem cells different from the stem cells of the first cultured meat, wherein
the first powdered cultured meat and the second powdered cultured meat are mixed with each other in a powdered form, and
the first powdered cultured meat and the second powdered cultured meat are prepared through the method for preparing powdered cultured meat according to claim 1.

9. The powdered cultured meat according to claim 8, which comprises 10 to 45 wt % of adipose stem cell-derived powdered cultured meat and 55 to 90 wt % of muscle stem cell-derived powdered cultured meat based on the total weight of the powdered cultured meat.

10. A food composition comprising the powdered cultured meat according to claim 6.

11. A specialized processed food comprising the powdered cultured meat according to claim 6.

12. The specialized processed food according to claim 11, wherein the specialized processed food is space food, C-ration, medical food or leisure food.
